(19) United States
(12) Patent Application Publication
(10) Pub. No.: US 2010/0256728 A1
(43) Pub. Date: Oct. 7, 2010

(54) Semi-Permiable Biodegradable Stent Graft and Uses Thereof

(75) Inventor: Susan Rea Peterson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/419,508

(22) Filed: Apr. 7, 2009

(51) Int. Cl. A61F 2/06

(52) U.S. Cl. 623/1.13; 623/1.46

(57) ABSTRACT

Described herein are biodegradable stent grafts and methods for treatment of early stage aneurysms and acute vessel injuries. The stent frameworks themselves are a biodegradable metal and the graft material is a semi-permeable, biodegradable porous fabric. The pores are sufficiently small to block the passage of monocytes through the semi-permeable fabric and sufficiently large to allow the passage of ions and small molecules through the semi-permeable fabric. The biodegradable stent grafts degrade completely leaving no permanent metal structures (or any structures at all) in the patient and therefore do not need to be later surgically removed or monitored.

### FIELD OF THE INVENTION

The present invention relates to biodegradable, semi-permeable stent grafts for vessel treatment, particularly vascular treatment and methods of using the stent grafts.

### BACKGROUND

Stent grafts have been developed to treat conditions of the vascular system. Stent grafts are primarily used to treat aneurysms of the vascular system and have also emerged as a treatment for a related condition, acute blunt aortic injury, where trauma causes damage to an artery.

Aneurysms arise when a thinning, weakening section of a vessel wall dilates and balloons. Actively treating such conditions is considered only when the vascular condition has developed to a point that is potentially life threatening to the patient. Aortic aneurysms (both abdominal and thoracic), for example, are treated when the vessel wall has expanded to at least 150% of its normal diameter. These dilated and weakened sections of vessel walls can and do burst, causing an estimated 32,000 deaths in the United States each year when not treated. Additionally, aneurysm rupture deaths are suspected of being underreported because sudden unexplained deaths, about 450,000 in the United States alone, are often simply misdiagnosed as heart attacks or strokes while many of them may be aneurysm ruptures which were not properly treated.

Surgeons in the United States treat approximately 50,000 abdominal aortic aneurysms each year, typically by replacing the abnormal section of vessel with a plastic or fabric graft in an open surgical procedure. A less-invasive procedure that has more recently been used is the placement of a stent graft at and across the aneurysm site. Stent grafts are tubular devices that span, or traverse the aneurysm site to provide support without replacing a section of the vessel. The stent graft, when placed within a vessel at an aneurysm site, acts as a barrier between blood flow and pressure and the weakened wall of a vessel, thereby decreasing pressure on the damaged portion. This less invasive approach to treat aneurysms decreases the morbidity seen with conventional aneurysm repair. Additionally, patients whose multiple medical comorbidities make them excessively high risk for conventional open surgical aneurysm repair are candidates for stent grafting.

While stent grafts represent alternatives and improvements over previously-used vessel treatment options, there are still risks associated with their use. One of these risks is associated with the timing of their use related to a patient's disease progression. Surgical treatment is often delayed by early attempts to treat and heal vascular complications using non-invasive and/or systemic means, therefore once surgical treatment (stent grafting) is performed, it is common for a patient to not fully attain the benefit of the treatment because the disease's progression has advanced beyond the point where it can be fully reversed so that a healthy native vessel is restored.

As such, stent grafts that can be deployed at an earlier stage of the progression of the vascular disease and/or even into smaller vessels that would otherwise be untreatable using a stent graft are described herein. Such treatment options and results would be beneficial to the field of vascular surgery. Such devices and methods will be described in greater detail in the following summary and detailed description.

### SUMMARY

Described herein are biodegradable stent grafts for treating body lumens, or vasculature, in need of medical intervention. The biodegradable stents are made up of one or more biodegradable struts interlinked to make a supportive, generally cylindrical structure and lined, covered or laminated with a semi-permeable fabric graft material which is also biodegradable.

Further, described herein are degradable stent grafts comprising: a stent comprising one or more biodegradable struts which may have reduced radial strength to minimize injury to the vessel wall; and a semi-permeable biodegradable fabric graft material attached to the reduced strength stent. In one embodiment, the stent graft further comprises a polymeric coating. In another embodiment, the stent graft further comprises a bioactive agent associated with the polymeric coating.

In still another embodiment, the stent graft has biodegradable struts comprising magnesium or an alloy of magnesium. In yet another embodiment, the stent graft has a radial strength of less than about 0.03 N/mm.

In one embodiment, the semi-permeable biodegradable fabric graft material associated with the stent comprises a loose weave configuration and can comprise one or more pores. The pores are sufficiently small to block the passage of monocytes through the semi-permeable biodegradable fabric graft material and are simultaneously sufficiently large to allow the passage of ions and small molecules through the semi-permeable biodegradable fabric graft material.

Further described herein are methods of treating an early stage aneurysm comprising the steps of: (a) identifying a patient population exhibiting a diseased vessel with an early stage aneurysm; (b) implanting a biodegradable stent graft comprising a semi-permeable biodegradable fabric graft material into the diseased vessel traversing the early stage aneurysm; (c) waiting for the early stage aneurysm to heal; (d) allowing a sufficient time for the biodegradable stent graft comprising a semi-permeable fabric graft material to biodegrade completely; and (e) treating the early stage aneurysm.

In one embodiment, the early stage aneurysm is defined to be a diseased vessel that has expanded to a diameter less than 150% of its normal diameter. In another embodiment, a sufficient time for stent graft degradation is less than 12 months or less than about 6 months.

Further still, described herein are methods of treating an acute vascular injury comprising the steps of: (a) identifying a patient population exhibiting a vessel with the acute vascular injury; (b) implanting a biodegradable stent graft comprising a semi-permeable biodegradable fabric graft material into the vessel traversing the acute vascular injury; (c) waiting for the acute vascular injury to heal; (d) allowing a sufficient time for the biodegradable stent graft comprising said semi-permeable fabric graft material to biodegrade completely; and (e) treating the acute vascular injury.

In one embodiment, the acute vascular injury is a blunt aortic injury. In another embodiment, the blunt aortic injury is a tear in a wall of the vessel.

In other embodiments, the semi-permeable biodegradable fabric graft material comprises one or more pores that are sufficiently small to block the passage of monocytes through the semi-permeable fabric and are simultaneously sufficiently large to allow the passage of ions and small molecules through the semi-permeable biodegradable fabric graft material.

In another embodiment, a sufficient time for stent graft degradation is less than 12 months or less than about 6 months.

### BRIEF DESCRIPTION OF THE DRAWINGS

FIG. 1 schematically illustrates a biodegradable stent graft.

FIG. 2 schematically illustrates a method of treating an early stage aneurysm using a biodegradable stent graft.

FIG. 3 schematically illustrates a method of treating an acute vessel injury using a biodegradable stent graft according to the present description

### DEFINITION OF TERMS

Before proceeding it may be useful to define many of the terms used to describe embodiments according to the present description. Words and terms of art used herein should be first defined as provided for in this specification, and then as needed as one skilled in the art would ordinarily define the terms.

As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

As used herein “biodegradable” refers to a composition that is biocompatible and subject to being broken down in vivo through the action of normal biochemical pathways. From time-to-time bioresorbable and biodegradable may be used interchangeably, however they are not coextensive. Biodegradable compositions may or may not be reabsorbed into surrounding tissues, however, all bioresorbable compositions are considered biodegradable. Biodegradable compositions are capable of being cleaved into biocompatible byproducts through chemical- or enzyme-catalyzed reactions.

As used herein “controlled release” refers to the release of a bioactive compound, or drug, from a medical device surface at a predetermined rate. Controlled release implies that the bioactive compound does not come off the medical device surface sporadically in an unpredictable fashion and does not “burst” off of the device upon contact with a biological environment (also referred to herein as first order kinetics) unless specifically intended to do so. However, the term “controlled release” as used herein does not preclude a “burst phenomenon” associated with deployment. In some embodiments according to the present description an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

As used herein “reduced strength” refers to the overall reduction in the stent graft's radial strength and radial stiffness when compared to conventional stent grafts used for treating advanced vessel complications, such as an abdominal aortic aneurysm. Radial strength is generally defined as the pressure which a stent graft exerts to the vessel or lumen into which it is implanted. If the radial strength of a stent graft is too low, the stent graft may not adhere well to the vessel wall after implantation within a vessel or body lumen. Therefore, a stent graft, or stent, must possess a minimum radial strength, which is dependent on the conditions into which it is being implanted, so that the forces acting on the stent graft do not overcome the radial strength and cause the stent graft to migrate. Conventional stent grafts have radial strengths greater than 0.03 N/mm. The radial strengths of the stent grafts described herein can be moderately low in comparison, typically less than about 0.03 N/mm. More preferably between about 0.02 N/mm and about 0.01 N/mm, or between about 0.01 N/mm and about 0.001 N/mm.

### DETAILED DESCRIPTION

Described herein are biodegradable stent grafts for treatment of body lumens, or vessels (e.g. blood vessels) in need of medical intervention. The stents are made up of one or more biodegradable struts interlinked to make a supportive, generally cylindrical structure. The cylindrical stent frameworks or structures are lined, covered or laminated with a semi-permeable fabric graft material used to partition the contents of the lumen being treated from the lumen wall in need of treatment.

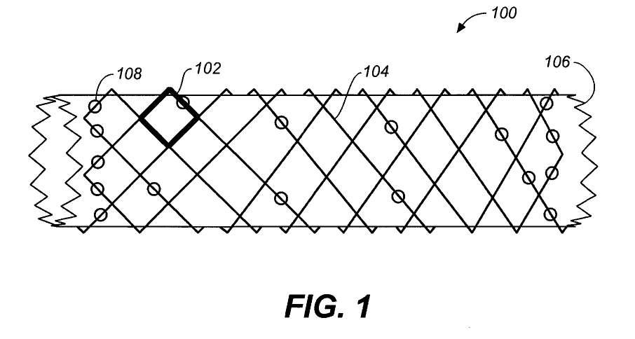

As illustrated in FIG. 1, stent graft 100 is assembled from one or more struts 102 to form a generally cylindrical stent structure 104. Struts 102 are made of a biocompatible material and can be tailored to degrade in vivo at a pre-determined rate. An exemplary biodegradable material used to form struts 102 and hence cylindrical stent structure 104 is magnesium and alloys thereof.

Magnesium and its alloys are biocompatible, biodegradable and easy to mechanically manipulate presenting an attractive solution for forming struts for biocompatible stent grafts. Magnesium also is useful in that it is radiopaque. Radiological advantages of magnesium include compatibility with magnetic resonance imaging (MRI), magnetic resonance angiography and computed tomography (CT). Vascular stent grafts comprising magnesium and its alloys are less thrombogenic than other bare metal stents. The biocompatibility of magnesium and its alloys stems from its relative non-toxicity to cells. Magnesium is abundant in tissues of animals and plants; specifically magnesium is the fourth most abundant metal in cells, the most abundant free divalent ion, and therefore, is deeply and intrinsically woven into cellular metabolism. Magnesium alloys which are biodegradable and suitable for forming biodegradable stent grafts according to the present description include alloys of magnesium with other metals including, but not limited to, aluminum and zinc. In one embodiment, the magnesium alloy comprises between about 1% and about 10% aluminum and between about 0.5% and about 5% zinc.

The magnesium alloys further include, but are not limited to, Sumitomo Electronic Industries (SEI, Osaka, Japan) magnesium alloys AZ31 (3% aluminum, 1% zinc and 96% magnesium) and AZ61 (6% aluminum, 1% zinc and 93% magnesium). The main features of these alloys include high tensile strength and responsive ductility. Tensile strength of typical AZ31 alloy is at least 280 MPa while that of AZ61 alloy is at least 330 MPa.

The biodegradable stent structures described herein can further be coated with a biocompatible polymer further controlling the stent graft's degradation rate. One or more coatings of a single polymer or two or more different polymer coatings or polymer mixtures can be used to control the degradation time. Further, the molecular weight of a polymer can be varied to achieve longer degradation times. As one skilled in the art is aware, high molecular weight polymers degrade over longer periods of time than similar lower molecular weight versions of the same polymer. Therefore, magnesium or magnesium alloys can be used to form a biodegradable stent and polymeric coatings can be coated onto the stent to lengthen the degradation time.

Exemplary biodegradable polymers useful for coating the biodegradable stent structures described herein include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid and combinations thereof.

The polymers above can be coated directly on the stent structures or can be applied over a primer layer such as parylene. Methods of coating bare metal stents are well known in the art and include spraying, dipping, rolling, painting, vapor deposition, sputtering and the like.

The biodegradable stent grafts described herein further include tubular graft material 106. Graft material 106 is preferably a woven fabric or porous polymer that is attached to cylindrical stent structure 104 at one or more connection point 108. Connection point 108 can include any means of connecting graft material 106 to cylindrical stent structure 104. Means for connection include sewing, laser welding, melting, weaving the fabric between the struts of cylindrical stent structure 104, bonding using an adhesive (e.g. an acrylate), and combinations thereof. Graft material 106 can be located on the inside or the outside of cylindrical stent structure 104.

If woven fabric is preferred, the woven fabric used for grafting onto the stent itself is a biocompatible and biodegradable fabric that is semi-permeable, preferably, a loose weave is used for the fabric. Varying the tightness of the fabric's weave allows a skilled artisan to attain a preferred opening, or pore size within the fabric.

If a porous polymer is preferred, the porous polymer used for grafting onto the stent itself is a biocompatible and biodegradable polymer that is semi-permeable. Producing a porous polymer is a method that is well known in the art. One exemplary method of providing pores in a polymer is to form the polymer with salt particles of known diameter. The diameter of the salt particles will translate into the diameter of the pores in the resulting polymer. Once a polymer is formed including a salt, the salt can be leached out of the polymer and pores remain in the spaces previously occupied by the salt particles.

The openings in the graft materials used herein are preferably less than about 10 μm in diameter, preferably between about 5 μm and about 8 μm. The nature of the opening described herein, whether they be opening in a fabric's weave or a pore produced in a polymer, the nature of the openings is such that they are sufficiently small to block the passage of monocytes through said semi-permeable fabric and sufficiently large to allow the passage of ions and small molecules through said semi-permeable fabric.

In other embodiments according to the present description, a highly loose weave fabric can be used as a graft material. This highly loose weave fabric can be coated with a porous polymer in order to achieve proper porosity of the graft material while providing a foundation for the polymer if needed. This arrangement of polymer coated fabric allows the skilled artisan to fine tune the porosity of a polymer that would otherwise not work as a grafting material. The polymeric coating can be added to the interior or exterior of the graft material.

Further, the polymers either used as a graft material alone or coated onto a fabric graft material can include one or more of the bioactive agents described above. The bioactive agents can be included on the inside or outside of the graft, or can be associated with both the inside and outside.

The biodegradable polymers associated with the biodegradable stent grafts described herein can further include a bioactive agent useful in local treatment of the injured or diseased vessel. Bioactive agents useful for this purpose include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention.

One exemplary anti-inflammatory bioactive agent useful in conjunction with the biodegradable stent grafts of the present description is SP600125, a C-Jun N-terminal kinase (JNK) inhibitor. The structure of SP600125 is as follows.

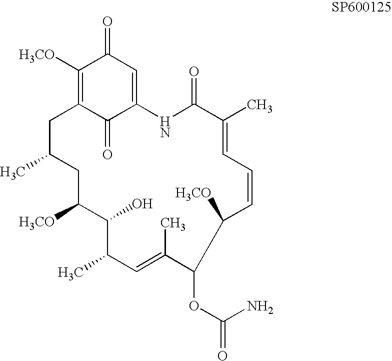

The biodegradable stent grafts described herein generally have a predetermined degradation time in vivo. This time is defined as a sufficient time for the stent graft to degrade completely leaving only a bare vessel. The sufficient time can be less than about 12 months, preferably less than about 6 months. Stent grafts described herein can even degrade completely in as short as 3 months. The needed degradation time is dependent on the individual, the severity of the condition being treated, the incorporated bioactive agents and the like. The devices described herein are manufactured in such a manner that each device will have a predetermined lifetime in vivo and can be tailored to achieve a necessary degradation time.

The biodegradable stent grafts described herein can have a reduced strength when compared to more commonly used stents or stent grafts. This reduced strength is generally a reduction in radial strength and stems from the fact that the biodegradable stent grafts described herein can be used for early stage aneurysm treatment and treatment of acute vessel injury wherein the vessel is not in need of the radial support of a severely diseased vessel. Further, if the biodegradable stent grafts described herein are used to treat acute vessel injuries, such as a tear, there is not a need to hold open a previously stenosed vessel. Therefore, the radial strengths can be moderately low compared to that of convention stent grafts, typically less than about 0.03 N/mm. More preferably the radial strengths may be between about 0.02 N/mm and about 0.01 N/mm, or between about 0.01 N/mm and about 0.001 N/mm.

The biodegradable stent grafts described herein can be used to treat early stage aneurysms. Aneurysms are commonly classified by how much expansion relative to a normal vessel has occurred. Commonly, stenting, either with a stent or a stent graft, is only performed when a vessel has expanded to greater than 150% its normal diameter. Early stage, in contrast, as used herein is defined as a vessel that has expanded less than 150% of its normal diameter. More preferably, the vessel is less than 140% of its normal diameter, or less than 130% of its normal diameter. The stent grafts described herein can also be used to treat early stage aneurysms wherein the vessel is expanded from about 100% to about 120% of its normal diameter.

The biodegradable stent grafts described herein can be used to treat vascular complications such as, but not limited to early stage aneurysms and acute vascular injuries. The following non-limiting methods further describe the usefulness of the present description over methods known in the art.

The first step in early aneurysm intervention involves identifying a patient population, or patient, exhibiting a diseased vessel with an early stage aneurysm. It is believed that if a population with early stage aneurysms is identified, the progression can be stopped and even fully revered. Without being bound to any particular theory, it is believed that at early stages of aneurysm progression, the biologic and physical properties of the vessel are very different than those at later stages which current stent grafts are deployed. In early stage aneurysms, the local vessel still has considerable amounts of smooth muscle cells and relatively healthy elastin content present. Therefore, early stage aneurysms have a greater chance of recovery than late stage aneurysms if aneurysm-progressing stimuli such as macrophages from the blood stream are prevented from reaching the early stage aneurysm.

The semi-permeable fabric graft materials used herein can help achieve this goal of early stage aneurysm treatment. The graft materials can allow small ion (e.g. through osmosis) and small nutrients to still reach the vessel wall by passing through the semi-permeable fabric, but can reduce the physical blood flow forces and screen out large cells such as monocytes. The screening out of monocytes prevents them from adhering to the vessel wall and differentiating into macrophages thereby continuing the aneurysm development cascade.

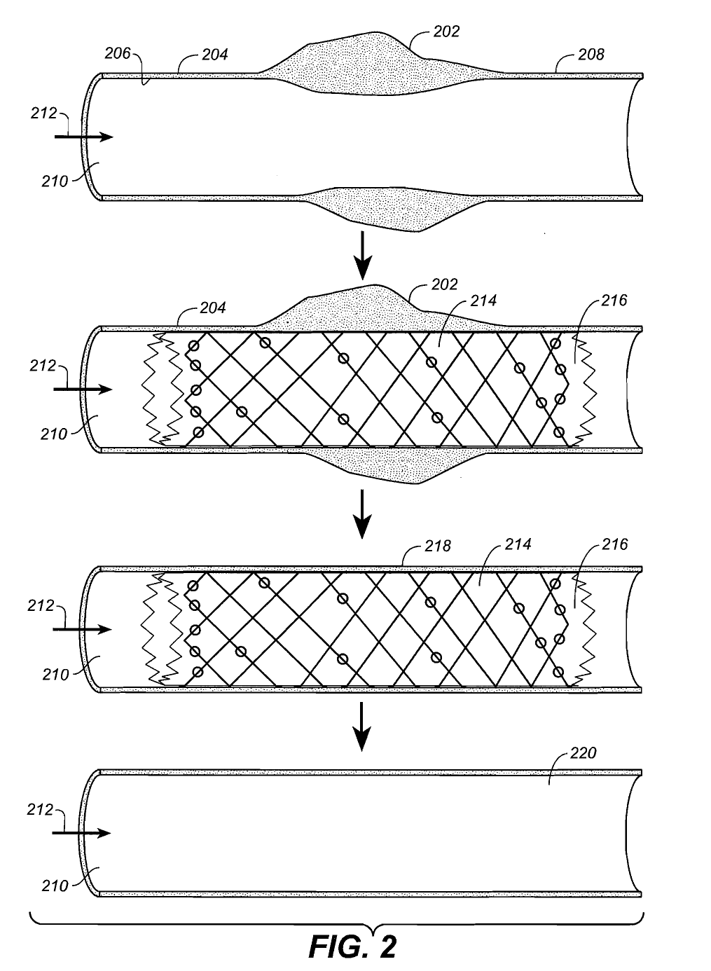

FIG. 2 outlines the general method of treating an early stage aneurysm according to the present description. As previously described, a population is identified wherein an early stage aneurysm 202 is located on or within vessel 204. Vessel 204 has an inner surface 206 and an outer surface 208 and an inner channel 210 wherein fluid, e.g. blood, can flow in direction 212.

Once the early stage aneurysm 202 is identified, a biodegradable stent graft 214 can be inserted into vessel 204 traversing the early stage aneurysm 202. Stent graft 214 is inserted using a catheter and can be either self expanding or expanded suing a balloon. Once expanded, stent graft 214 supports vessel 204 at early stage aneurysm 202 and seals off early stage aneurysm 202 from blood flow (e.g., direction 212) using biodegradable, semi-permeable graft material 216. Biodegradable, semi-permeable graft material 216 allows ions and nutrients from the blood to pass through, but does not allow larger species, such as cells or monocytes, to pass through to the early stage aneurysm 202.

After the early stage aneurysm 202 is allowed to regenerate leaving healed vessel segment 218, a sufficient amount of time is allowed for stent graft 214 including biodegradable, semi-permeable graft material 216, to break down by the bodies natural pathways and be eliminated completely. A sufficient time can be less than about 12 months, preferably less than about 6 months. The devices described herein are manufactured in such a manner that each device will have a predetermined lifetime in vivo and can be tailored to achieve a necessary lifetime.

In some embodiments according to the present description, biodegradable, semi-permeable graft material 216 degrades and is bioabsorbed faster than the stent structure it is associated with. In such a device, the graft material can provide its partitioning effect for the blood from the early aneurysm site and then degrade while the stent support structure remains for a longer period of time to help support the newly regenerated vessel wall. After a given time, the stent structure can degrade as well. In such an embodiment, the graft material can degrade completely within about 3 months and the stent structure in about 6 months, or the graft material can degrade completely within about 6 months and the stent structure in about 12 months. In other embodiments, the graft material and the stent structure can degrade at the same rate, providing partitioning and support for the life of the entire device.

Once the early stage aneurysm 202 is completely healed and sufficient time has passed for stent graft 214 including biodegradable, semi-permeable graft material 216 to completely degrade, a newly regenerated, stent less vessel 220 remains and the patient has been treated.

The biodegradable stent grafts described herein can also be used to treat acute vascular injury. Examples of acute vascular injury include, but are not limited to blunt vessel injuries such as tears in a vessel wall, holes in a vessel wall, bruising in a vessel wall and the like. Such injuries commonly require immediate medical attention and, in most cases surgical remedies. The biodegradable stent grafts described herein can be particularly appealing to treat a younger cohort of patients experiencing such acute injuries, because after the stent graft has done its job and an appropriate amount of time has passed, the stent graft will be eliminated from the body and only a healed, healthy vessel will remain requiring no need for follow-up intervention to remove an implanted device.

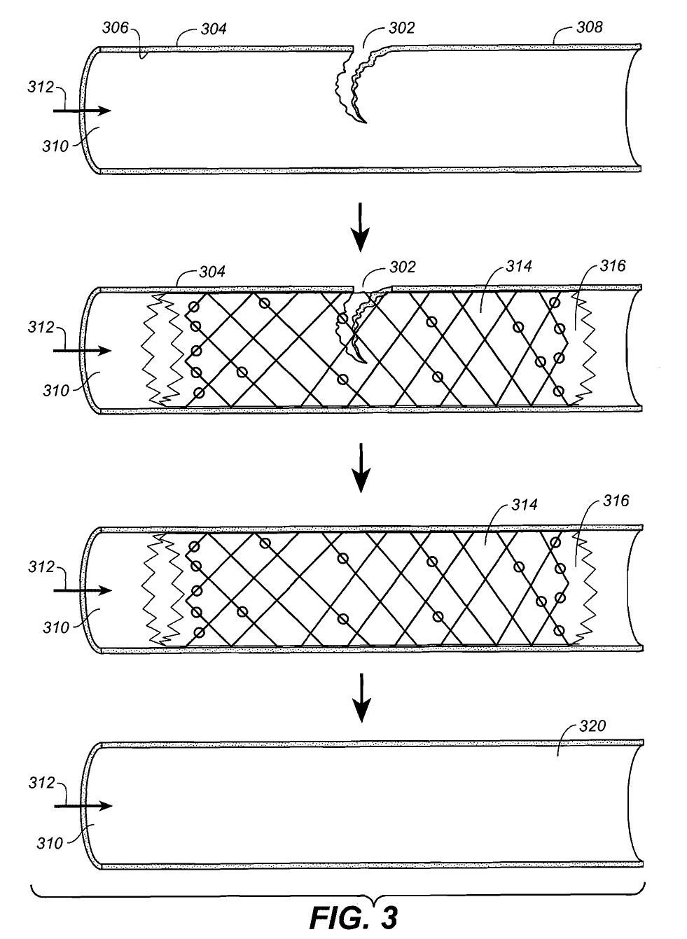

FIG. 3 outlines the general method of treating an acute vascular injury as described herein. As previously described, a population is identified, in this case having an aortic tear 302 located on vessel 304. Vessel 304 has an inner surface 306 and an outer surface 308 and an inner channel 310 wherein fluid, e.g. blood, can flow in direction 312.

Once the aortic tear 302 is identified, biodegradable stent graft 314 can be inserted into vessel 304 traversing aortic tear 302. Stent graft 314 is inserted using a catheter and can be either self expanding or expanded using a balloon. Once expanded, stent graft 314 supports vessel 304 at aortic tear 302 and seals off aortic tear 302 from blood flow (e.g., direction 312) using biodegradable, semi-permeable graft material 316. Biodegradable, semi-permeable graft material 316 allows ions and nutrients from the blood to pass through, but does not allow larger species, such as cells or monocytes to pass through to the aortic tear 302. This partitions off aortic tear 302 from the unwanted constituents of the blood and hemodynamic forces themselves. This aids in the healing process.

After aortic tear 302 is allowed to regenerate leaving healed vessel segment 318, a sufficient amount of time is allowed for stent graft 314 including biodegradable, semi-permeable graft material 316, to break down by the bodies natural pathways and be eliminated completely. A sufficient time can be less than about 6 months, preferably less than about 3 months when the device is used to treat an acute vessel injury. The devise described herein are manufactured in such a manner that each device will have a predetermined lifetime in vivo and can be tailored to achieve a necessary lifetime.

Once aortic tear 302 is completely healed and sufficient time has passed for stent graft 314 including biodegradable, semi-permeable graft material 316 to completely degrade, a newly regenerated, stent-less vessel 320 remains and the patient has been treated.

The use of the biodegradable stent grafts according to the present description is particularly beneficial to a younger population experiencing an acute vessel injury or an early stage aneurysm, because the vessels in these individuals are generally otherwise healthy. Therefore, the biodegradable stents can aid in vessel healing and then be removed by natural pathways instead of surgical removal. The biodegradable stent graft can also be useful for older patients as well, because it stifles the need for surgical removal of an implanted device in a population susceptible to surgical complications.

### EXAMPLE 1

### Treating an Early Stage Aneurysm

A 39 year old male is admitted to the hospital with shortness of breath and chest pain. The patient is diagnosed as having an early stage aneurysm, but is otherwise in good health. It is decided by the attending physician to treat the patient with a biodegradable stent graft. The stent itself is made of a magnesium and zinc alloy and the porous graft is made of polycaprolactone and impregnated with SP600125. The stent graft is implanted thereby traversing the early stage aneurysm using a standard catheter.

After the implantation surgery, the patient recovers and returns home two days after the procedure. After 6 months, the patient returns to his physician for a follow-up examination. The physician indicates that the early stage aneurysm has completely healed and the stent graft has begun to degrade naturally. Upon returning to the physician after one year, the stent graft is completely degraded and all that remains is a healthy vessel.

### EXAMPLE 2

### Treating an Early Stage Aneurysm in a Feeble Patient

A 75 year old female is admitted to the hospital with shortness of breath. The patient is diagnosed as having an early stage aneurysm, and it is determined that open surgery to fix the vessel is not an option. It is decided by the attending physician to treat the patient with a biodegradable stent graft. The stent itself is made of a magnesium and zinc alloy and the porous graft is made of polycaprolactone and impregnated with SP600125. The stent graft is implanted thereby traversing the early stage aneurysm using a standard catheter.

After 6 months, the patient returns to her physician for a follow-up examination. The physician indicates that the early stage aneurysm has completely healed alleviating the need for further intervention. The stent graft then begins to degrade and after a year is completely eliminated, saving the feeble patient from having to deal with any complications and life long monitoring related to having a permanent metal device in her body.

### EXAMPLE 3

### Treating an Acute Vessel Injury

A 25 year old male is admitted to the emergency room after a near fatal motorcycle accident. The patient is diagnosed as having a slight tear in his aorta. It is decided by the attending physician to treat the patient with a biodegradable stent graft. The stent itself is made of a magnesium and zinc alloy and the porous graft is made of polycaprolactone and impregnated with SP600125. The stent graft is implanted thereby traversing the early stage aneurysm using a standard catheter.

After 3 months, the patient returns to his physician for a follow-up examination. The physician indicates that the slight tear in the aorta has completely healed alleviating the need for further intervention. The stent graft then begins to degrade and after 6 months is completely eliminated, saving the patient from having to again undergo a surgical procedure to remove the stent graft or any complications associated with having a permanent implanted medical device.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by embodiments according to the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of embodiments according to the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing embodiments according to the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate embodiments according to the invention and do not pose a limitation on their scope.

Groupings of alternative elements or embodiments according to the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments according to the invention are described herein. Of course, variations on these described embodiments will become apparent to those of ordinary skill in the art upon reading the foregoing description. It is expected that skilled artisans will employ such variations as appropriate, and that embodiments according to the invention will be practiced otherwise than specifically described herein. Moreover, any combination of the above-described elements in all possible variations thereof is encompassed unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments according to the invention disclosed herein are illustrative. Other modifications may be employed. Thus, by way of example, but not of limitation, alternative configurations according to the present invention may be utilized in accordance with the teachings herein. Accordingly, embodiments according to the present invention are not limited to that precisely as shown and described.

I claim:

1. A degradable stent graft comprising:
a stent comprising one or more biodegradable struts; and
a semi-permeable biodegradable fabric graft material attached to said stent.

2. The degradable stent graft according to claim 1 further comprising a polymeric coating.

3. The degradable stent graft according to claim 2 further comprising a bioactive agent associated with said polymeric coating.

4. The degradable stent graft according to claim 1 wherein said biodegradable struts comprise magnesium.

5. The degradable stent graft according to claim 4 wherein said magnesium is an alloy of magnesium.

6. The degradable stent graft according to claim 1 wherein said stent comprises a reduced radial strength of less than about 0.03 N/mm.

7. The degradable stent graft according to claim 1 wherein said semi-permeable biodegradable fabric graft material comprises a loose weave configuration.

8. The degradable stent graft according to claim 1 wherein said semi-permeable biodegradable fabric graft material comprises one or more pores.

9. The degradable stent graft according to claim 8 wherein said pores are sufficiently small to block the passage of monocytes through said semi-permeable biodegradable fabric graft material.

10. The degradable stent graft according to claim 8 wherein said pores are sufficiently large to allow the passage of ions and small molecules through said semi-permeable biodegradable fabric graft material.

11. A method of treating an early stage aneurysm comprising the steps of:
(a) identifying a patient population exhibiting a diseased vessel with an early stage aneurysm;
(b) implanting a biodegradable stent graft comprising a semi-permeable biodegradable fabric graft material into said diseased vessel traversing said early stage aneurysm;
(c) waiting for said early stage aneurysm to heal;
(d) allowing a sufficient time for said biodegradable stent graft comprising a semi-permeable biodegradable fabric graft material to biodegrade completely; and
(e) treating said early stage aneurysm.

12. The method according to claim 11 wherein said early stage aneurysm comprises a diseased vessel that is expanded less than 150% of its normal diameter.

13. The method according to claim 11 wherein said semi-permeable biodegradable fabric graft material comprises one or more pores that are sufficiently small to block the passage of monocytes through said semi-permeable biodegradable fabric graft material and sufficiently large to allow the passage of ions and small molecules through said semi-permeable biodegradable fabric graft material.

14. The method according to claim 11 wherein said sufficient time is less than 12 months.

15. The method according to claim 14 wherein said sufficient time is less than 6 months.

16. A method of treating an acute vascular injury comprising the steps of:
(a) identifying a patient population exhibiting a vessel with said acute vascular injury;
(b) implanting a biodegradable stent graft comprising a semi-permeable biodegradable fabric graft material into said vessel traversing said acute vascular injury;
(c) waiting for said acute vascular injury to heal;
(d) allowing a sufficient time for said biodegradable stent graft comprising said semi-permeable biodegradable fabric graft material to biodegrade completely; and
(e) treating said acute vascular injury.

17. The method according to claim 16 wherein said acute vascular injury is a blunt aortic injury.

18. The method according to claim 17 wherein said blunt aortic injury is a tear in a wall of said vessel.

19. The method according to claim 11 wherein said semi-permeable biodegradable fabric graft material comprises one or more pores that are sufficiently small to block the passage of monocytes through said semi-permeable biodegradable fabric graft material and sufficiently large to allow the passage of ions and small molecules through said semi-permeable biodegradable fabric graft material.

20. The method according to claim 16 wherein said sufficient time is less than 12 months.

21. The method according to claim 14 wherein said sufficient time is less than 6 months.
